(19) United States
(12) Patent Application Publication
(10) Pub. No.: US 2016/0074199 A1
(43) Pub. Date: Mar. 17, 2016

(54) Reinforced lower limb orthotic brace

(71) Applicant: Wade Bader, Lutz, FL (US)

(72) Inventor: Wade Bader, Lutz, FL (US)

(21) Appl. No.: 14/121,522

(22) Filed: Sep. 15, 2014

(51) Int. Cl. A61F 5/01

(52) U.S. Cl. CPC A61F 5/0127

(57) ABSTRACT

A reinforced lower limb orthotic brace to restrict the movement of the calf and foot of a patient including an upper load bearing member having a lower foot support member affixed thereto and a reinforcing member extending along the upper load bearing member.

### BACKGROUND OF THE INVENTION

1. Field of the Invention

A reinforced lower limb brace to limit or restrict the movement of the lower limb and foot of a patient.

2. Description of the Prior Art

Numerous efforts have been made to design and manufacture a light weight, durable orthotic brace. Many use new materials or combinations thereof. Examples of these devices are discussed below.

U.S. Pat. No. 7,749,423 discloses a method of producing an orthotic brace or prosthetic device comprising the steps of creating an anatomical mold or cast of that portion of the patient's anatomy upon which the orthotic brace or prosthetic device is to be applied, applying at least one layer of epoxy resin over at least a portion of the anatomical mold, applying at least one layer of carbon graphite fabric on at least a portion of the layer of epoxy resin, placing the anatomical mold with the epoxy resin and carbon graphite fabric in an enclosure having an interior, heating the interior of the enclosure to melt the epoxy resin and reducing the pressure in the interior of the enclosure causing the melted epoxy resin to impregnate the carbon graphite fabric, and allowing the melted epoxy resin to harden in the carbon graphite fabric forming the orthotic brace or prosthetic device.

U.S. Pat. No. 6,146,344 describes a lower limb orthotic brace to control the movement of the lower limb of a patient comprising a contoured orthotic contact shell and an integral load-bearing frame. The contoured orthotic contact shell comprises at least one layer of fabric impregnated with a hardened structural resin forming an inner orthotic contact surface conforming to the outer surface of the lower limb of the patient. The integral load-bearing frame comprises at least one reinforcing member extending along the contoured orthotic contact shell including a plurality of bundled continuous composite fibers woven in a multidirectional tubular braid impregnated with a hardened structural resin with at least one side of the braid in contact with the contoured orthotic contact shell along the entire length of the braid such that the hardened resins impregnating the fabric of the contoured orthotic contact shell combine with the resins impregnating the braid thereby forming a continuous integral structural matrix encapsulating both the braid and the contoured orthotic contact shell.

U.S. Pat. No. 5,817,041 shows a lower limb orthotic comprising a foot orthotic, a pair of lateral supporting members, a detached or removable anterior support member, a posterior support member, and strips of composite materials to resist plantar flexion, dorsiflexion and various of movements of the foot and ankle. The lateral supporting members further having strips of composite materials having fibers orientation substantially parallel to the length wise axis of the lateral supporting members. The foot orthotic and posterior supporting member further have composite strips extending across the bottom portion of the foot orthotic and rear side of the posterior supporting member respectively.

U.S. Pat. No. 5,693,007 teaches a method and apparatus for forming a custom fit knee orthotic including forming a flat preassembled knee orthotic of rigid carbon fiber and plastic resin composite material connected in a flat, rigid state by joint mechanisms.

U.S. Pat. No. 5,624,386 relates to an orthopedic brace using the shaped composite bars as integral components, and a method of thermoshaping the composite bars for use as filled components in the orthopedic brace are provided. The composite bars contain multiple fiber layers oriented in at least two directions with respect to the bar length to provide high flexural and torsional strength. The novel method of thermo-shaping yields improved results over prior thermo-shaping methods by providing an uncomplicated shaping capability while maintaining the structural and mechanical properties of the composite bar.

U.S. Pat. No. 5,573,501 shows a laminated, plastic orthotic device custom fitted to a wearer's extremity, typically a leg, wherein the orthotic includes a indicia bearing layer viewable on the outer surface of the orthotic. In one preferred embodiment, the device is fabricated from multiple layers of woven, reinforced and non-reinforced fabrics; adhesives, curable plastic resins and an image bearing layer. In other preferred embodiments, the device is fabricated of thermosetting plastic materials and an image bearing layer. The image layer may be comprised of woven fabric or non-woven materials which include drawings, insignias, photographs, textures or combinations thereof.

U.S. Pat. No. 5,154,690 discloses a supporter for mounting on a limb in position to cover an affected body portion to constrain and limit movement thereof comprising a tubular stretchable elastic material forming a main body and reinforcing member comprising an interlining laminated to a surface member which acts to restrict the elongation of said interlining.

U.S. Pat. No. 4,813,090 shows a hybrid composite material constructed from woven reinforced fabrics of Kevlar 49 and biaxially reinforced fibers of graphite and E-Glass bonded together with a thermosetting adhesive, for the application of custom orthotics. The material interlocks different fibers at different locations of the orthotic in order to take advantage of the unique properties of each reinforcing fibers. The resulting material creates a hybrid composite that exhibits a balance of properties, in both the longitudinal and transverse directions, unavailable with any single reinforcing fibers. A method of orthotic manufacturing involves a hand flat-up and forming procedure. The constructed composite material is formed and pressed on a prescribed cast, then allowed to cure under pressure. The post-cured material is stripped from the cast, then cut and ground and finally finished to the required orthotic. The produced orthotic combines the desired feature of a thin section with excellent combination of properties relevant to custom orthotics. Such properties include light weight, high strength and modulus, excellent resistance to impact, fatigue, and creep, and outstanding ability to damp vibrations.

U.S. Pat. No. 4,672,955 teaches the thigh and calf bands of a knee orthotic formed of a layer or layers or curable composite material. The sidebars are integral with the thigh and calf bands in that they are simply extensions of the layers forming the thigh and calf bands. The sidebars are cured prior to fitting the thigh and calf bands about the leg, but the thigh and calf bands are left incurred so that they may be formable about the leg and cured in place thereon.

U.S. Pat. No. 5,158,530 discloses a method of applying an orthopedic cast to an injured body portion consisting of braiding a tubular cast, in situ, around the injured body portion. A braiding machine applies the tubular braid of fibrous casting yarns around the injured body portion to form a braided cast which conforms to the shape of the injured body portion. The braided cast is then coated with a matrix material, which provides the necessary rigidity to the cast, yet allows the cast to breathe.

U.S. Pat. No. 3,682,163 shows a light weight, semi-rigid snap on orthopedic splint having a flexible sheet plastic main body laminated to a thick spongy layer and equipped with long shallow channels for the insertion of flexible reinforcing splints. The edges of the main body are provided with a suitable adjustable fastener as one having a multiplicity of L-shaped ribs which interlock selectively in different overlapped positions as necessary to form a snug fit with a limb or any part of the body and notched crosswise of the ribs to adapt the splint for assembly about a tapering portion of the body as a limb.

U.S. Pat. No. 3,040,740 discloses a prefabricated article for incorporation under surgical casts and molds comprising a plurality of superimposed concentrically disposed seamless tubular layers of loosely knit cotton cloth. Each layer of cloth is formed of intercoupled threads in co-acting relationship with each other with certain threads intersecting other threads at juncture points forming oblique angles and defining open interstices therebetween with the distance between adjacent juncture points along any thread being greater than the diameter of any of the threads so that when pulled the layers of cloth will deform without stretching the intercoupled threads by having the juncture points which are generally aligned to the direction of pull separating a substantial amount in relation to each other and the juncture points which are generally aligned transversely to the direction of pull covering a substantial amount in relation to each other.

U.S. Pat. No. 5,020,523 relates to a splint device for splinting and immobilizing the lower leg, ankle, and foot including L-shaped inner core member having a slightly cupped heel portion, a horizontal basal portion extending frontally from the heel portion and a slightly curved vertical portion extending upwardly from the heel portion. A flexible foam cover is disposed on the core member to form a body of the splint device which is positionable against the lower leg, heel, and plantar surface of the foot, the cover being sized and configured to fully conceal the core member therewithin.

U.S. Pat. No. 5,282,483 shows a immobilizing apparatus for an injured body part including a rigid plate conforming to the injured body part and having two opposite faces, upon one of which the injured body part is provided; a limiting unit to limit the lateral movement of the injured body part on the rigid plate; an adjustable member by which the overall length of the rigid plate can be adjusted; and a fastening member to fasten the injured body part, immobilizing the injured body part on the rigid plate.

U.S. Pat. No. 5,593,383 discloses a securing apparatus for an ankle and foot orthotic brace. The brace is used for supporting and selective immobilization of a patient's ankle and foot. The brace has a multiple part L-shaped construction with a contoured leg support portion and a foot portion having a resilient interconnecting heel portion connected therebetween. The securing apparatus includes a foot pad with aperture tabs extending therefrom and being part of the foot portion. A fabric foot engagement enclosure is provided which secures to the foot pad by a plurality of adjustable fastening straps engaged through said apertures to the aperture tabs.

U.S. Pat. No. 5,799,659 relates to a device for treatment of foot and ankle conditions comprising a rigid, molded shell having a generally U-shaped cross-sectional configuration and a flat foot bed covered by a soft fabric covering. Removable and interchangeable foot bed wedge insert permits the angle of dorsiflexion, plantarflexion, inversion and eversion to be varied.

U.S. Pat. No. 5,897,515 shows a ankle-foot orthotic of a carbon fiber reinforced material having low weight is carried on the front of the lower leg, extending over the lateral ankle and preventing plantar flexion. The ankle-foot orthotic comprises a frame of thin flexible material extending over the front of the lower leg, anterior of the lateral ankle and beneath the sole of the foot and a supporting portion of rigid material extending over a narrow part of the front of the lower leg, anterior of the lateral ankle and beneath the part of the sole of the foot. The orthotic includes a fastening means for fastening the orthotic to the leg. In a preferred embodiment the orthotic comprises a substantially inflexible reinforcement element and a tough flexible element, the reinforcement element extending over a narrow part of substantially the whole frame and the flexible part extending over a substantial part of the sole of the foot. The frame is preferably made of thin flexible fiber glass reinforced plastic resin material. The reinforcement element is made of rigid carbon fiber reinforced plastic resin material. With the tough flexible element is preferably made of aramid fiber reinforced plastic resin.

U.S. Pat. No. 6,146,349 describes a copolymer thermoplastic natural foot orthotic for supporting and controlling the movement of a lower extremity. The orthotic is fabricated by forming a positive mold of the lower extremity and modifying the positive mold in predetermined locations to accomplish the type of lower extremity control desired. A strip of heated thermoformable copolymer material is strategically positioned around predetermined locations on the positive mold for providing increased support for the natural foot orthotic at these predetermined locations. A sheet of heated thermoformable copolymer material is then drape mold around the positive mold and copolymer strip wherein the copolymer sheet is vacuum sealed to conform to the shape of the positive mold. The copolymer sheet and copolymer strip are integrally formed into a single supporting structure. After the thermoformed copolymer sheet and strip have cooled, trimlines are made in the thermoformed copolymer sheet depending upon the lower extremity control desired wherein all excess material outside of the trimlines is removed. Utilizing modifications to the positive mold and strategically positioned trimlines in conjunction with the reinforcing copolymer strip, a natural foot orthotic is achieved that is lighter, cosmetically superior, dynamic and durable.

U.S. Pat. No. 6,676,618 teaches a ankle-foot orthotic made integrally from a thin, shaped lightweight material. The orthotic comprises a flat foot-supporting member for extending beneath the sole of a foot of a user, a calf abutment member for abutting the calf of the user, a narrow connecting member extending from the foot-supporting member at a location on one side, preferably the outer side, of the ankle of the user to the calf abutment member so as to interconnect the foot-supporting member and the calf abutment member, and a releasable fastener for fastening the calf abutment member to a leg of the user. The orthotic may be made by a method, in which a thermoplastic material with reinforcing fibers is arranged between opposite plastic films or foils in a desired mutual arrangement. Thereafter, the space defined between the opposite films is sealed, and air or gas is then removed from the sealed space so as to compact the material arranged therein and so as to form a blank. The blank thus formed is thereafter heated to a plasticizing temperature and formed into the desired shape.

G.B. Pat. No. 2,188,550 shows a calf band which is to pass around the leg, has a central portion connected to the upper end portion of a posterior leaf spring and two lateral portions which are to overlap in front, of the leg and which are connected together by suitable means. The central portion provides a cushion between the upper end portion and the calf.

Additional examples of the prior art are found in U.S. Pat. No. 5,458,656 and U.S. Pat. No. 6,394,971.

While some of the prior art may contain some similarities relating to the present invention, none of them teach, suggest or include all of the advantages and unique features of the invention disclosed hereafter.

### SUMMARY OF THE INVENTION

The present invention relates to a reinforced lower limb orthotic brace to restrict the movement of the lower limb or calf and foot of a patient.

The reinforced lower limb orthotic brace comprises an upper load bearing member having a lower foot support member affixed to the lower portion thereof. A first or front transition area or zone and a second or rear transition area or zone are formed at the front intersection of the upper load bearing member and the mid-section of the lower foot support member and at the intersection of the load bearing member and the mid-section of the lower foot support member.

Significantly, the first or front intersection and the second or rear intersection between the upper load bearing member and the lower foot support member are relatively weak with respect to the entire lower limb orthotic brace.

The upper load bearing member is reinforced by the integral reinforcing member comprising an outer sleeve or casing having at least two inner cores disposed thereon.

The integral reinforcing member extends from the upper portion of the load bearing member and expands or diverges laterally outward to overlap the front transition area or zone and the rear transition area or zone to a portion of the lower foot support member affixed to the upper load bearing member.

Similarly, each inner core extends from the upper portion of the load bearing member diverging laterally outward relative to each other such that one inner core overlays the front transition area or zone and the other inner core overlays the rear transition area or zone to the portion of the lower foot support member affixed to the upper load bearing member.

The outer sleeve or casing may comprise a plurality of bundles of continuous composite fibers woven together in a tubular multidirectional braid.

Each inner core comprises an outer casing of multidirectional braid and an inner element of epoxy resin disposed within.

Of course, the epoxy resin and the multidirectional braid may be placed side by side or sandwiched similar in fashion to the configuration shown in FIGS. 12 and 13 of U.S. Pat. No. 7,749,423. Regardless of configuration of the inner cores, the reinforcing member and the lower limb orthotic brace including the upper load bearing member and a portion of the lower foot support member on the sides and bottom thereof adjacent the front transition area or zone and the rear transition area or zone are integrally bonded in a manner or method similar to that discussed in U.S. Pat. No. 7,749,423.

The invention accordingly comprises the features of construction, combination of elements, and arrangement of parts which will be exemplified in the construction hereinafter set forth, and the scope of the invention will be indicated in the claims.

### BRIEF DESCRIPTION OF THE DRAWINGS

For a fuller understanding of the nature and object of the invention, reference should be had to the following detailed description taken in connection with the accompanying drawings in which:

FIG. 1 is a side view of the reinforced lower limb orthotic brace of the present invention.

FIG. 2 is a top cross-sectional view of the narrow lower interconnecting portion of the upper load bearing member of the present invention taken along line 2-2 of FIG. 1.

FIG. 3 is a bottom view of the lower foot support member of the present invention.

Similar reference characters refer to similar parts throughout the several views of the drawings.

### DETAILED DESCRIPTION OF THE PREFERRED EMBODIMENT

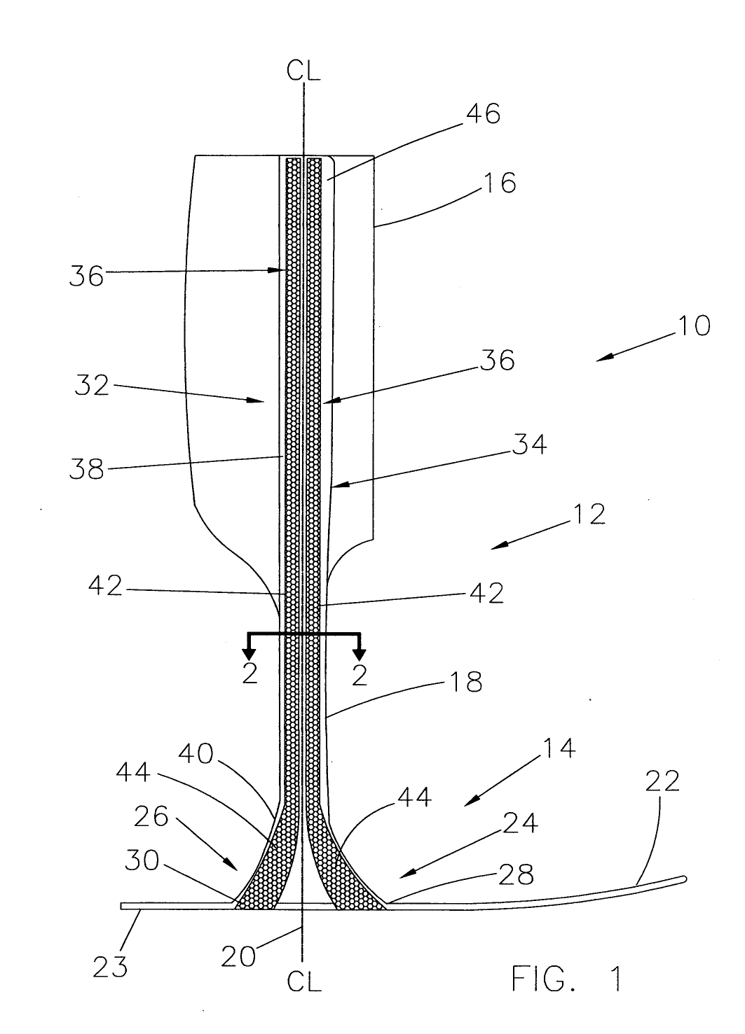

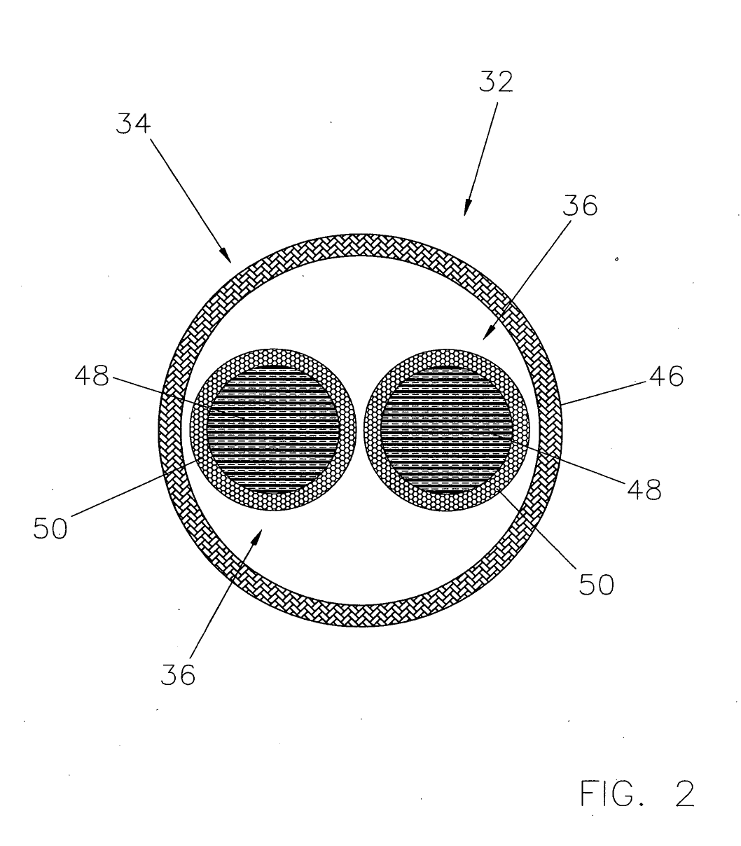

As shown in FIGS. 1 and 2, the present invention relates to a reinforced lower limb orthotic brace generally indicated as 10 to limit or restrict the movement of the lower limb or calf and foot of a patient.

As seen in FIG. 1, the reinforced lower limb orthotic brace 10 comprises a substantially vertical upper load bearing member generally indicated as 12 having a lower foot support member generally indicated as 14 affixed to the lower portion thereof.

As seen in FIG. 1, the substantially vertical upper load bearing member 12 comprises an upper calf engaging portion 16 and a narrow lower intermediary portion 18 connecting the upper calf engaging portion to the lower foot support member 14.

As best seen in FIG. 1, the lower foot support member 14 comprises a substantially horizontal flat median or major foot support portion or section 20 to support the arch area of the foot, an upwardly curved or arched forefoot portion or section 22 to support the forward part of the foot and toes, and a substantially horizontal flat rear portion or section 23 to support the heel.

As best seen in FIG. 1, a first or front transition area or zone generally indicated as 24 and a second or rear transition area or zone generally indicated as 26 are formed at the first or front intersection 28 of the first or front portion of the narrow lower interconnecting portion 18 of the substantially vertical load bearing member 12 and the mid-section of the substantially horizontal flat medial or major foot support portion or section 20 of the lower foot support member 14 and at the second or rear intersection 30 of the second or rear portion of the narrow lower interconnecting portion 18 of the substantially vertical load bearing member 12 and rear section of the substantially horizontal flat medial or major foot support portion or section 20 respectively.

Significantly, the first or front intersection 28 and the second or rear intersection 30 between the narrow lower interconnecting portion 18 of the substantially vertical upper load bearing member 12 and the lower foot support member 14 are relatively weak with respect to the entire lower limb orthotic brace 10.

As shown in FIGS. 1 and 2, the upper calf engaging portion 16 and the narrow lower interconnecting portion 18 of the substantially vertical upper load bearing member 12 are reinforced by a reinforcing member generally indicated as 32 comprising an outer sleeve or casing generally indicated as 34 having at least two inner cores each generally indicated as 36 disposed therein. The reinforcing member 32, upper load bearing member 12 and lower foot support member 14 are integrally formed together.

As seen in FIG. 1, a substantially vertical upper sleeve portion 38 extends from the upper portion of the upper calf engaging portion 16 of the substantially vertical upper load bearing member 12 and substantially through the center line L of the narrow lower interconnecting portion 18 thereof expanding laterally outward to overlay the first or front transition area or zone 24 and the second or rear transition area or zone 26 to a portion of the substantially horizontal flat medial or major foot support portion or section 20 of the lower foot support member 14.

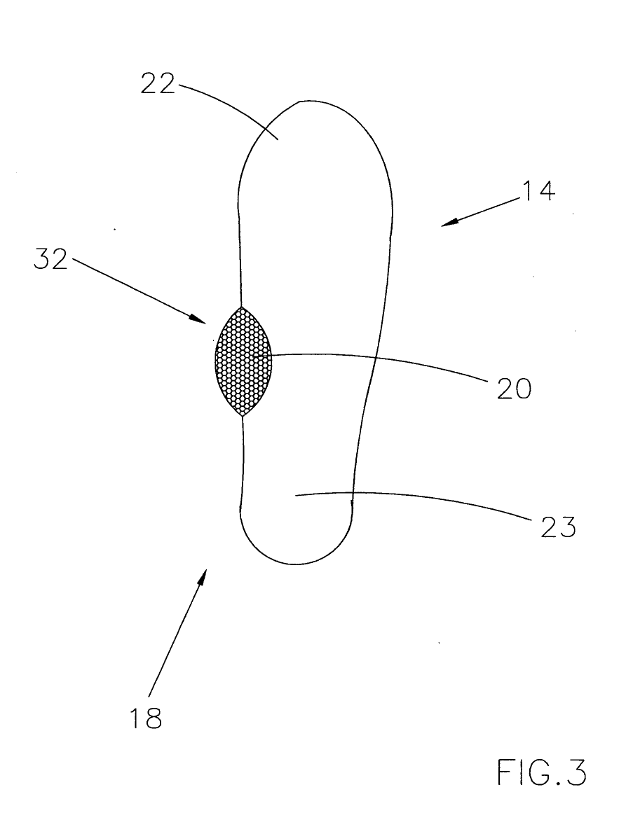

Similarly as shown in FIG. 1, each inner core 36 extends from the upper calf engaging portion 16 of the upper portion of the substantially vertical upper load bearing member 12 on either side of the center line CL of the narrow lower interconnecting portion 18 thereof and diverges laterally outward relative to each other such that one inner core 36 overlays the first or front transition area or zone 24 and the other inner core 36 overlays the second or rear transition area or zone 26 to a portion of the substantially horizontal flat medial or major foot support portion or section 20 of the lower foot support member 14 (see FIG. 3).

The outer sleeve or casing 34 may comprise a plurality of bundles of continuous composite fibers woven together in a tubular multidirectional braid 46.

The multidirectional braid may include individual continuous composite fibers of graphite, carbon, Kevlar or other similar commercially available materials.

As seen in FIG. 2, each inner core 36 comprises an inner element or rod of epoxy resins 48 disposed with an outer casing of multidirectional braid 50. Of course, the epoxy resin 48 and the multidirectional braid 50 may be placed side by side or sandwiched similar in fashion to the configurations shown in FIGS. 12 and 13 of U.S. Pat. No. 7,749,423. Regardless, the configuration of the inner core 36, the reinforcing member 32 and the lower limb orthotic brace 10 including the substantially vertical upper load bearing member 12 and a portion of the lower foot support member 14 on the sides and bottom thereof adjacent the first or front transition area or zone 24 and the second or rear transition area or zone 26 are integrally bonded using a method similar to that disclosed in U.S. Pat. No. 7,749,423.

Although not shown, a flex calf strip or the like can be used to secure the reinforced lower limb orthotic brace to the patient's calf as depicted in FIG. 1 of U.S. Pat. No. 6,146,344.

It will thus be seen that the objects set forth above, among those made apparent from the preceding description are efficiently attained and since certain changes may be made in the above construction without departing from the scope of the invention, it is intended that all matter contained in the above description or shown in the accompanying drawing shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

Now that the invention has been described,

What is claimed is:

1. A reinforced lower limb orthotic brace to restrict the movement of the lower limb or calf and foot of a patient including an upper load bearing member to engage the calf of the patient having a lower foot support member to support the foot of a patient affixed thereto and a reinforcing member including at least one inner core element disposed within an outer sleeve extending along at least the major portion of said upper load bearing member expanding laterally adjacent the intersection of said upper load bearing member and said lower front support member to reinforce a front transition area or zone and a rear transition area or zone formed at the intersection of a first or front portion of said upper load bearing member and said lower front support member and the intersection of said second or rear portion of said upper load bearing member and said lower foot support member respectively.

2. A reinforced lower limb orthotic brace to restrict the movement of the calf and foot of a patient including an upper load bearing member having a lower foot support member affixed thereto and a reinforcing member extending along the upper load bearing member.

3. The reinforced lower limb orthotic brace of claim 2 wherein said upper load bearing member comprises an upper calf engaging portion and a narrow lower intermediate portion connecting said upper calf engaging portion to said lower foot support member.

4. The reinforced lower limb orthotic brace of claim 3 wherein said lower foot support member comprises a median or major foot support portion or section to support the arch area of the foot, a forefoot to support the forward part of the foot and toes, and a rear portion or section to support the heel.

5. The reinforced lower limb orthotic brace of claim 4 wherein a first or front transition area or zone and a second or rear transition area or zone are formed at a first or front intersection of said first or front portion of said narrow lower interconnecting portion of said load bearing member and the mid-section of said medial or major foot support portion or section of said lower foot support member and at a second or rear intersection of said second or rear portion of said narrow lower interconnecting portion of said load bearing member and rear section of said medial or major foot support portion or section respectively.

6. The reinforced lower limb orthotic brace of claim 5 wherein said upper calf engaging portion and said narrow lower interconnecting portion of said upper load bearing member are reinforced by a reinforcing member integrally formed therewith.

7. The reinforced lower limb orthotic brace of claim 6 wherein said reinforcing member comprises an outer sleeve or casing having at least two inner cores disposed therein.

8. The reinforced lower limb orthotic brace of claim 7 wherein said upper sleeve portion extends from the upper portion of said upper calf engaging portion of said upper load bearing member and substantially through the center line of said narrow lower interconnecting portion thereof expanding laterally outward to overlay said first or front transition area or zone and said second or rear transition area or zone to a portion of said medial or major foot support portion or section of said lower foot support member.

9. The reinforced lower limb orthotic brace of claim 8 wherein each said inner core extends from said upper calf engaging portion of said upper portion of said upper load bearing member on either side of the center line of said narrow lower interconnecting portion thereof and diverges laterally outward relative to each other such that said one inner core overlays said first or front transition area or zone and said other inner core overlays said second or rear transition area or zone to a portion of said medial or major foot support portion or section of said lower foot support member.

10. The reinforced lower limb orthotic brace of claim 9 wherein said outer sleeve or casing said comprise a plurality of bundles of continuous composite fibers woven together in a tubular multidirectional braid.

11. The reinforced lower limb orthotic brace of claim 10 wherein each said inner core comprises an inner element or rod of epoxy resins disposed with an outer casing of multidirectional braid.
